(19) United States
(12) Plant Patent Application Publication
(10) Pub. No.: US 2009/0265822 P1
(43) Pub. Date: Oct. 22, 2009

(54) Tiarella Plant Named 'Snow Blanket'

(50) ;

(75) Inventor: Daniel M. Heims, Portland, OR (US)

Correspondence Address: KLARQUIST SPARKMAN, LLP, 121 SW SALMON STREET, SUITE 1600, PORTLAND, OR, 97204, US

(73) Assignee: Terra Nova Nurseries, Inc.

(21) Appl. No.: 12/148,437

(22) Filed: Apr. 17, 2008

(51) Int. Cl. A01H 5/00

(52) U.S. Cl. PLT/263.1

(57) ABSTRACT

A new and distinct cultivar of Tiarella plant characterized by its compact, slow spreading habit, pink buds to white flowers, palmately divided leaves with a dark starry blotch in the center and excellent vigor.

### BOTANICAL DENOMINATION

Tiarella spp.

### VARIETY DESIGNATION

‘Snow Blanket’

### BACKGROUND OF THE INVENTION

The present invention relates to a new and distinct cultivar of a hardy herbaceous perennial of the genus Tiarella, and known by the cultivar name ‘Snow Blanket’. The genus Tiarella is a member of the Saxifragaceae family.

The new cultivar originated as a cross between unknown parents. This new Tiarella was one of many seedlings grown from select experimental and proprietary interspecific hybrids and species that were mass pollinated in the greenhouse in Canby, Oreg.

### SUMMARY OF THE INVENTION

This plant is uniquely characterized by the following:

Compact habit.

Slow spreading habit.

Palmately divided leaves with a dark blotch at the leaf center.

Pink flower buds and white flower color.

Excellent vigor.

The new variety has been reproduced only by asexual propagation (division and micropropagation). Each of the progeny exhibits identical characteristics to the original plant. Asexual propagation by division and micropropagation using standard techniques with terminal and lateral shoots as done in Canby, Oreg., shows that the foregoing characteristics and distinctions come true to form and are established and transmitted through succeeding propagations. The present invention has not been evaluated under all possible environmental conditions. The phenotype may vary with variations in environment without a change in the genotype of the plant.

### BRIEF DESCRIPTION OF THE DRAWINGS

FIG. 1 shows a two-year-old Tiarella ‘Snow Blanket’ in spring in the garden in Canby, Oreg.

FIG. 2 shows the same plant in the summer.

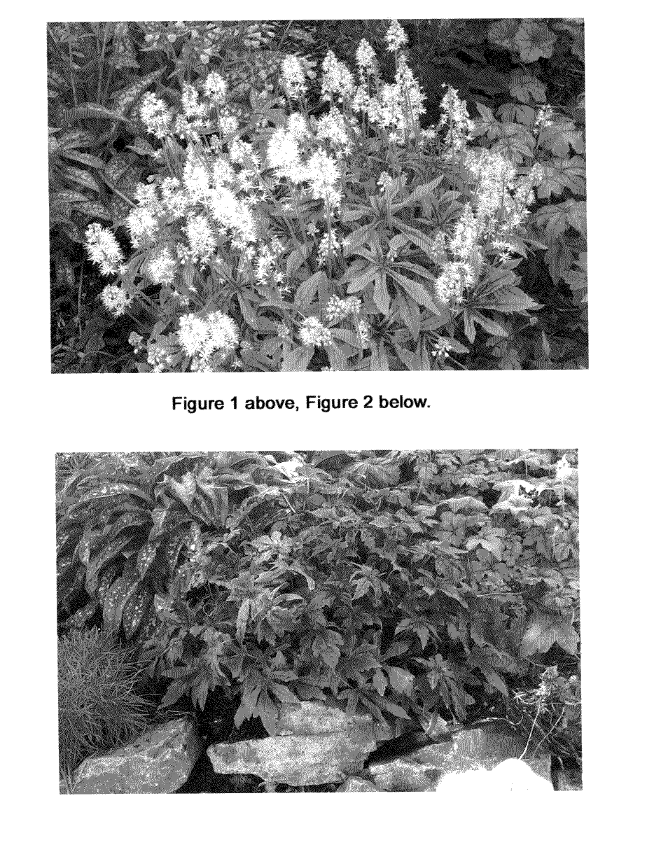

### DETAILED PLANT DESCRIPTION

The following is a detailed description of the new Tiarella hybrid based on observations of two-year-old plants grown outside in a one gallon container in the open air with 50% shade cover and added irrigation in the summer months in Canby, Oreg. Canby is Zone 8 on the USDA Hardiness map. Temperatures range from a high of 95 degrees F. in August to an average low of 32 degrees F. in January. Normal rainfall in Canby is 42.8 inches per year. The color descriptions are all based on The Royal Horticultural Society Colour Chart.
Plant:
   Type.—herbaceous perennial.
   Hardiness.—USDA Zones 4 to 9.
   Form.—spreading mound.
   Size.—35 cm wide and 20 cm high from the soil to the top of the foliage.
   Vigor.—excellent.
   Roots.—fibrous, freely branching, fine, and white in color. Roots develop easily from crown divisions.
Foliage:
   Type.—simple.
   Arrangement.—rosette.
   Shape.—ovate overall.
   Lobing.—5 to 7 narrow lobes, palmately deeply parted; terminal lobe is the longest and lanceolate, entire, the top ¾ lobed; lateral lobes are oblong to oblanceolate with entire margins on bottom ¼ of the lobes, crenate to lobed on top ¾.
   Margins.—unevenly crenate.
   Venation.—palmate.
   Apex.—mucronulate.
   Base.—cordate.
   Blade size.—grows to 9.5 cm long and 7.5 cm wide.
   Texture.—rough.
   Surface.—hispid top and bottom, matte.
   Petiole description.—grows to 19.5 cm long, hirsute, Yellow Green 148A with a brown tint at the base, Greyed Orange 177A.
   Leaf color.—Spring topside — closest to Yellow Green 147A but a little darker, with a small dark blotches along the main veins, Greyed Purple 183A Summer and winter topside — closest to Yellow Green 147A but a little darker, with brown blotching almost to the leaf margin. The dark pattern is Brown 200A Bottom side year round — Yellow Green 147B.
Inflorescence:
   Type.—raceme.
   Flower number.—about 55 per raceme.
   Number of flowering stems in the spring flush.—over 100.
   Bloom period.—April to May with sporadic rebloom throughout the summer.
   Peduncle.—unbranched and without leafy bracts, grows to 32 cm tall and 3 mm in diameter, hispid, color varies from Greyed Green 197A to Yellow Green 148A.
   Pedicel description.—7 mm long and 2 mm wide, hispid, Greyed Orange 177D.
   Lastingness.—a raceme blooms for about 3 weeks.
Flower Bud:
   Size.—4 mm deep and 2.5 mm wide.
   Description.—ovoid, down facing until open.
   Color.—pink, Red Purple 65B.
Flower:
   Type.—perfect, actinomorphic, sepals petaloid.
   Shape.—rotate.
   Flower size.—5 mm wide and 5 mm deep including stamens and pistil.
   Corolla description.—5 petals, lanceolate with a clawed base, reflexed when mature, 2.5 mm long and 0.5 mm wide, entire, acuminate, White 155D inside and outside.
   Calyx description.—rotate, 5 lobed, spreading 6 mm wide and 1 mm deep, petaloid, parted almost to the base, oblanceolate, White 155A inside and outside, lobes 3 mm long and 1 mm wide, entire, obtuse.
   Stamen.—10, conspicuously exerted, filaments 5 mm long, White 155B, anthers undehisced Orange Red 31C, pollen color Orange 26B.
   Pistil.—White 155A overall, 2.5 mm long, ovary 1.4 mm long, style 1 mm long, stigma 0.5 mm.
   Fragrance.—Sweet.
Fruit:
   Type.—A one-celled capsule, beak-like, composed of two unequal parts.
   Size.—0.8 cm long.
   Color.—Brown 200C.
Seeds:
   Color.—Black 202A.
   Shape.—Minute, oval, 1 mm long.
   Fertility.—Low.
Pest and disease resistance: This new hybrid shows good mildew tolerance, the main problem for Tiarella. No major pest problems. It is susceptible to root weevils like all Tiarella.
Comparisons with similar Tiarella:Compared with Tiarella ‘Jeepers Creepers’ (U.S. Plant Pat. No. 13,437), Tiarella ‘Snow Blanket’ has leaves that are much more cut and cut palmately rather than pinnately and the foliage is a deeper green. Compared to Tiarella ‘Ninja’ (U.S. Plant Pat. No. 11,168), this new cultivar is has a spreading rather than a clumping habit.

I claim:

1. A new and distinct cultivar of Tiarella plant as herein illustrated and described.
